(19) United States
(12) Patent Application Publication
(10) Pub. No.: US 2026/0286419 A1
(43) Pub. Date: Sep. 24, 2026

(54) DETECTING CONCENTRATION OF SMALL MOLECULES IN SOLUTION

(71) Applicants: Benjamin Siedlarz, Springfield, VA (US); Samir Zuberi, Springfield, VA (US)

(72) Inventors: Benjamin Siedlarz, Springfield, VA (US); Samir Zuberi, Springfield, VA (US)

(21) Appl. No.: 19/082,583

(22) Filed: Mar. 18, 2025

(51) Int. Cl. C12Q 1/32; C12Q 1/00

(52) U.S. Cl. CPC C12Q 1/32; C12Q 1/004; C12Q 1/005

(57) ABSTRACT

A method for conducting a small molecule assay has steps for selecting a BIO-FET having a redox mediator bound to an exposed surface of a graphene layer from a stored quantity, applying an enzyme with sterile phosphate buffer solution (PBS) to the redox mediator-bound graphene layer, agitating the BIO-FET for a time period, allowing the enzyme to fully bind at the graphene surface, applying a DC voltage across the first and second electrodes, applying a digital voltmeter between the first electrode and a metal bottom layer of the BIO-FET, covering the graphene layer with a subject small molecule sample, allowing a time for small molecules molecules in the subject sample to fully react with the enzyme; and recording a reading of the DVM as an indication of concentration of the small molecule in the subject sample.

### BACKGROUND OF THE INVENTION

### 1. Field of the Invention

The present invention is in the technical field of diagnostic testing and relates more specifically to electrochemical testing of solution samples to detect concentration of small molecules, such as lactates.

### 2. Description of Related Art

The most effective method of diagnosing cancer at the time of filing the instant application is awareness of common side-effects and medical protocols in practice. Detection, for example, of an unusual swelling and a report of pain may lead to discovery of an instance of a cancerous tumor in a subject. There are known in the art a few diagnostic tests for specific cancers, and lactate as a cancer indicator in a subject urine sample may be detected via chromatography (qualitative) but this is expensive and requires a lab for testing and is not as precise as a quantitative assay.

What is needed is a universal, highly accurate, and inexpensive test for the presence of cancer in subjects, a test that can be administered in real time to reduce mortality rate of the disease and increase effectiveness of chemotherapeutics. At the time of filing the instant application there are no universal cancer detection tests. The tests that are available are specific and limited to a few tumor types and genomes.

### BRIEF SUMMARY OF THE INVENTION

A method for preparing a BIO-FET having a graphene layer between a first and a second electrode for a aqueous small molecule assay is provided, having steps for wetting an exposed surface of the graphene layer with a redox mediator solution, and allowing time for the redox mediator to bind to the graphene layer. In one embodiment the redox mediator is nicotinamide adenine dinucleotide (NAD+). Also, in one embodiment the method further comprises rinsing the BIO-FET with a sterile phosphate buffer solution (PBS), drying the BIO-FET after rinsing, and packaging and storing the dried BIO-FET for later use.

In another embodiment a method for conducting a small molecule assay is provided, having steps for selecting a BIO-FET having a redox mediator bound to an exposed surface of a graphene layer from a stored quantity, applying an enzyme with sterile phosphate buffer solution (PBS) to the redox mediator-bound graphene layer, agitating the BIO-FET for a time period, allowing the enzyme to fully bind at the graphene surface, applying a DC voltage across the first and second electrodes, applying a digital voltmeter between the first electrode and a metal bottom layer of the BIO-FET, covering the graphene layer with a solution of a subject small molecule, allowing a time for lactate molecules in the subject solution to fully react with the enzyme, and recording a reading of the DVM as an indication of concentration of the small molecule in the subject solution.

In one embodiment the redox mediator is nicotinamide adenine dinucleotide (NAD+), the solution of a subject small molecule is a subject urine sample, and the enzyme is an isoform of lactate dehydrogenase. Also, in one embodiment the method further comprises, after the agitation step and before the step applying the DC voltage across the electrodes, rinsing the BIO-FET with sterile PBS solution. Also, in one embodiment the method further comprises, after the step selecting a BIO-FET having nicotinamide adenine dinucleotide (NAD+) and before applying a lyophilized lactate dehydrogenase (LDHB) solution, recording a DVM reading as a baseline reading. In one embodiment the isoform of lactate dehydrogenase is LDHB. And in one embodiment the method further comprises executing a mobile application on a cellular telephone, connecting to a website through the mobile application, the website providing an interactive interface on a display of the cellular telephone enabling entry of the DVM reading, and entering the DVM reading in the interactive interface.

In one embodiment, in response to the entry of the DVM reading, information is displayed regarding lactate concentration in the subject urine sample, and optional meanings of the concentration. Also, in one embodiment the method further comprises comparing the DVM reading with one or more standard curves. In one embodiment the method further comprises determining by the comparing whether or not that the subject has lactate acidosis, and should be further evaluated.

In one embodiment of the invention an apparatus for conducting a small molecule assay is provided, comprising a field-effect transistor (FET) having a first and a second electrode and a metal bottom layer, a layer of graphene deposited on an upper surface of the FET between and in contact with the first and the second electrodes, a redox mediator bound to sites on the graphene layer, an enzyme bound to the redox mediator on the graphene layer, a DC power supply adapted to connect across the first and the second electrodes, and a digital voltmeter adapted to connect across the first electrode and the metal bottom layer of the BIO-FET. A sample solution containing an unknow concentration of the small molecule applied over the graphene surface causes molecules in the sample to interact with the enzyme, causing a DC voltage to appear across the first electrode and the metal bottom layer, measurable by the DVM as an indication of concentration of the small molecule in the sample solution.

In one embodiment of the apparatus the redox mediator is nicotinamide adenine dinucleotide (NAD+). Also, in one embodiment enzyme is an isoform of lactate dehydrogenase. Also, in one embodiment the DC power supply is battery powered and comprises first and second cables with contact mechanisms adapted to mate with contact mechanisms on each of the first and second electrodes. Also, in one embodiment the contact mechanisms on the cables are cylindrical pins and the contact mechanisms on the first and second electrodes are bores with a snug fit to the pins.

In one embodiment the DVM comprises a first cable with a first contact mechanism adapted to mate with a second contact mechanism at the first electrode, and a second cable with a third contact mechanism adapted to mate with a fourth contact mechanism at the metal bottom layer. Also, in one embodiment the contact mechanisms on the cables are cylindrical pins and the contact mechanisms on the first electrode and the metal bottom layer are bores with a snug fit to the pins.

In one embodiment of the invention a kit for determining concentration of small molecules in a subject sample is provided, comprising a biological field-effect transistor (BIO-FET) having first and second electrodes, a metal bottom layer, a layer of graphene deposited on an upper surface of the FET between and in contact with the first and the second electrodes and a redox mediator on the graphene layer, a battery enabled DC power supply configured to produce a DC voltage, the DC power supply having a first conductor ending in a first attachment interface, and a second conductor ending in a second attachment interface, a digital voltmeter having a display, a first cable with a first contact mechanism adapted to mate with a second contact mechanism at the first electrode, and a second cable with a third contact mechanism adapted to mate with a fourth contact mechanism at the metal bottom layer, a packet of dry enzyme powder, a capped container of phosphate buffer solution (PBS) mixed with DI water, a capped container of sterile phosphate buffer solution (PBS), and a plastic or glass pipette.

In one embodiment the redox mediator is nicotinamide adenine dinucleotide (NAD+) and the enzyme is an isoform of lactate dehydrogenase. In one embodiment the pipette is marked at a position indicating a specific volume. And in one embodiment the kit further comprises an instruction booklet having instructions for use of the kit in administering a test.

### BRIEF DESCRIPTION OF THE SEVERAL VIEWS OF THE DRAWINGS

FIG. 1 is a perspective view of a biological field effect transistor (BIO-FET) useful in an embodiment of the present invention.

FIG. 2 is a side elevation view of the BIO-FET of FIG. 1 with a surface treated with a redox mediator in an embodiment of the invention.

FIG. 3 is a side elevation view of the BIO-FET of FIG. 2 with a power supply and a voltage sensor added in an embodiment of the invention.

FIG. 4 is a diagrammatic illustration of a portion of the BIO-FET of FIG. 3 illustrating chemical reactions in an embodiment of the invention.

FIG. 5 is a flow diagram illustrating steps in a method for binding a redox mediator to graphene.

FIG. 6 is a flow diagram illustrating a method for determining lactate concentration in a urine sample.

FIG. 7 is an illustration of a do-it yourself (DIY) kit for lactate determination in a urine sample.

### DETAILED DESCRIPTION OF THE INVENTION

The present invention provides a method for measuring concentration of small molecules in a solution using biological mechanisms including enzymes and a redox mediator such as (NADH/NAD). By a small molecule the inventor means a molecule of relatively low molecular weight, such as ≤1000 daltons. A redox mediator is a molecule, ion, or compound that can be reversibly oxidized and reduced during a chemical reaction, acting as an intermediate electron carrier to facilitate electron transfer between different reactants, essentially to shuttle electrons between the reactants and to accelerate the overall reaction process.

In one embodiment the method of the invention the electrochemically active redox mediator is linked to a surface of graphene, a carbon nanostructure, or other conductive material, organic or inorganic, electrically associated with a field effect transistor such as a tungsten disulfide (WoS2), tungsten diselenide (WSe2), or graphene-based FET. The method further comprises linking an enzyme on or around the electrochemically active redox mediator on a surface of the field effect transistor. The method of the invention includes the described system being interacted with an isolated aqueous sample of the subject small molecules to either reduce or oxidize the redox mediator, the reduction or oxidation measured through the associated FET as an electrical, optical, thermal, or pH signal. The invention can be adapted for assaying a large number of metabolites through the chemical nature of the redox meditator and enzyme (multiple binding partners). An important use of the invention is to assess, through determination of elevated lactate in urine, a probability of cancer in the subject, to motivate further and specific tests for presence of cancer.

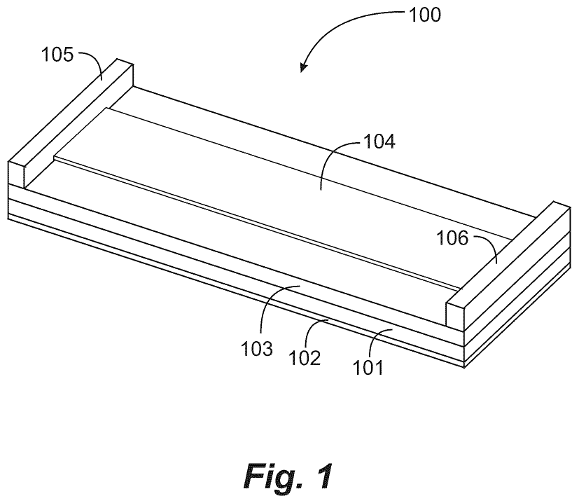

In a specific example of the invention a graphene-based BIO-FET is employed having a layer of graphene between a source and a drain electrode, which electrodes are both metal. FIG. 1 is a diagram of a BIO-FET apparatus 100 useful in an embodiment of the present invention, comprising a silicon substrate 101 having an upper layer 103 of silicon dioxide (SiO₂) and an underlayer of metal 102. A metal source electrode 105 and a metal drain electrode 106 are implemented on the BIO-FET, spaced apart on the SiO₂ layer, with a layer of graphene 104 deposited between the electrodes, in contact with both electrodes. BIO-FETs are well known in the art and are manufactured by also well-known semiconductor manufacturing techniques. The skilled person will realize that the BIO-FET illustrated might be formed in other geometries.

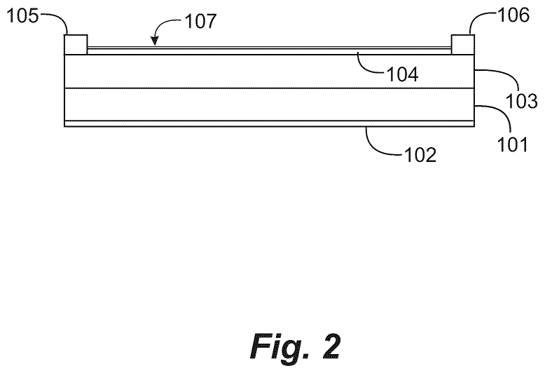

In one embodiment of the invention the redox mediator is nicotinamide adenine dinucleotide (NAD+), the oxidized form of NADH. In an initial step prior to instituting a diagnostic test on a subject BIO-FETs as in FIG. 1 are treated with NAD+ to bind NAD+ to carboxyl impurities at the graphene surface, forming what is termed a reduced graphene oxide FET (rGO-FET) as seen in FIG. 2 having a surface 107 bound with NAD+. This may be accomplished by wetting the surface of the graphene layer with a solution of NAD in PBS solution, allowing a time period for binding, which may be facilitated by an acid catalyst or heating, and perhaps then rinsing with such as a sterile phosphate buffer solution (PBS). A plurality of FETs with the graphene surfaces bound with NAD+ may be placed in protective packaging and stored for later use in diagnostic tests.

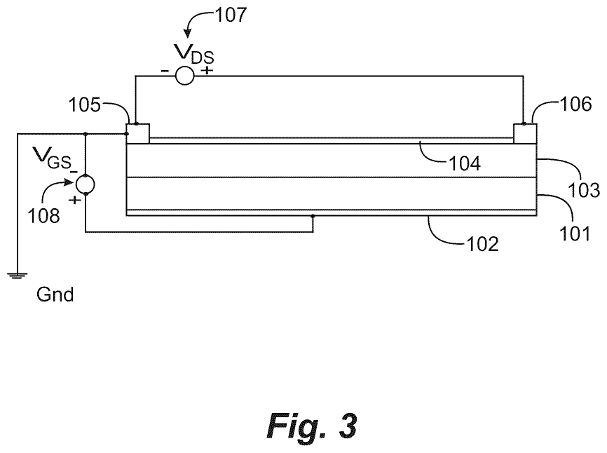

FIG. 3 illustrates the Bio-FET of FIG. 2 with addition of a DC voltage V_{DS} applied by a power supply 108 across the electrodes and a voltage sensor 109 implemented to measure DC voltage V_{GS} across the transistor, between source electrode 105 and metal underlayer 102. These additions are made in a step in a process of use of the apparatus described more fully below.

To perform a test a user selects a sensor device that has the graphene surface treated with NAD+ that has been stored for later use as described above. The next step is to rehydrate the surface of the BIO-FET with a solution of an isoform of lactate dehydrogenase with room temperature sterile PBS and add the mixed solution of the isoform, which may be LDHB, in PBS to the surface of the graphene sensor dropwise, making sure to cover the entire sensor area. The LDHB binds to NAD in milliseconds, depending on temperature, so a one-minute wait time while agitating the device will suffice. Optionally excess solution may be rinsed from the apparatus with, for example, sterile PBS solution.

At this point the sensor is nearly ready for a subject sample, however a base line reading to measure resistance is required before adding a subject sample. The baseline reading needs to be done with NAD+:rGO only optionally in PBS or dry conditions. Depending on size of the BIO-FET only approximately 10-50 ul of sample will be needed to cover the graphene surface of the device. Once the subject sample has been added dropwise to cover the graphene surface, which needs be in a temperature range of 25 to 37 degrees Celsius, the LDHB will take about 5 minutes to fully react with lactate in the subject sample.

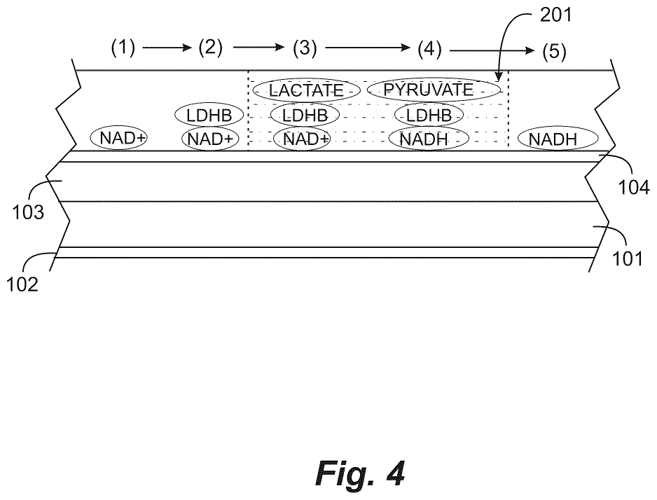

FIG. 4 is a diagrammatic illustration of a portion of the BIO-FET of FIG. 3 illustrating chemical reactions over time in an embodiment of the invention. The diagram shows chemical composition and reaction from left to right timewise labeled (1), (2), (3), (4) and (5). At time (1) the subject sample has not been added, and NAD+ is bound to the surface. The base line reading is to be taken at this time. At time (2) the graphene surface has been covered with a solution of LDHB, leaving LDHB bound to the NAD+. After time (2) a subject urine sample is added, and at time (3) there is lactate in the urine sample which may interact with the LDHB. At time (4) the lactate molecule is oxidized to pyruvate and the NAD+ at the surface is reduced to NADH, which is NAD plus a hydrogen molecule. At time (5) the FET has been rinsed with a PBS solution and NADH remains at the graphene surface.

The skilled person will understand that there will be a plurality of lactate molecules in the subject's sample and the description above is to explain what happens with just one. After waiting for the reactions to occur, the electrical characteristics are measured once more determining change in resistance and voltage which will be compared to a base line. The greater the reduction in resistance the more NADH is bound to the graphene and the higher the lactate concentration. In practice a standard curve may be compared against patient samples to reduce risk of inaccurate results.

In a typical subject approximately 83% of lactate is metabolized in the liver and kidney resulting in a small but measurable portion of lactate excreted in the urine (mmol/L to nmol/L) 17%. Healthy patients have a range of blood lactate level of 0.5 to 2 mmol/L and urine lactate levels will be correlated to blood lactate levels. Levels higher than 2.5 mmol/L are considered key indications hyperlactatemia a frequently encountered metabolic alteration in critically ill patients and should urge patients to seek further medical diagnostic care.

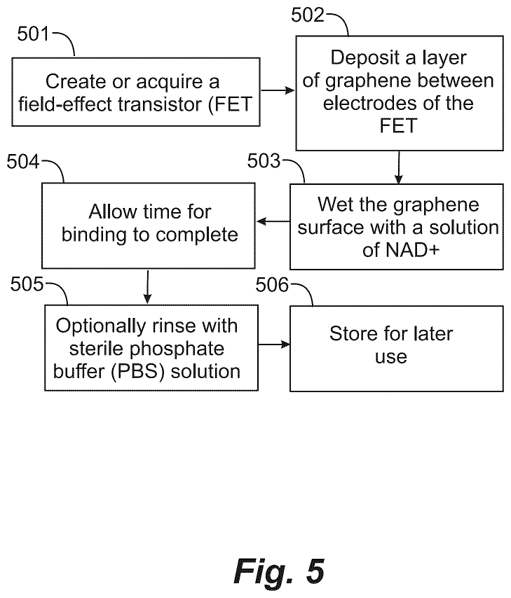

FIG. 5 is a flow diagram of steps in a process of preparing a BIO-FET prior to subject sample. At step 501 a field-effect transistor (FET) is created or acquired. At step 502 a layer of graphene is deposited between electrodes of the FET, making a BIO-FET. At step 503 the graphene layer is wet with a solution of NAD+ dropwise, covering the entire surface of the graphene layer. At step 504 time is taken for the binding of the NAD+ to the graphene to be complete. At step 505 the BIO-FET may optionally be rinsed with a sterile solution of PBS and then dried. At step 506 the BIO-FET is stored for later selection and use. It may be assumed that a significant plurality of BIO-FETs may be prepared and stored for later use.

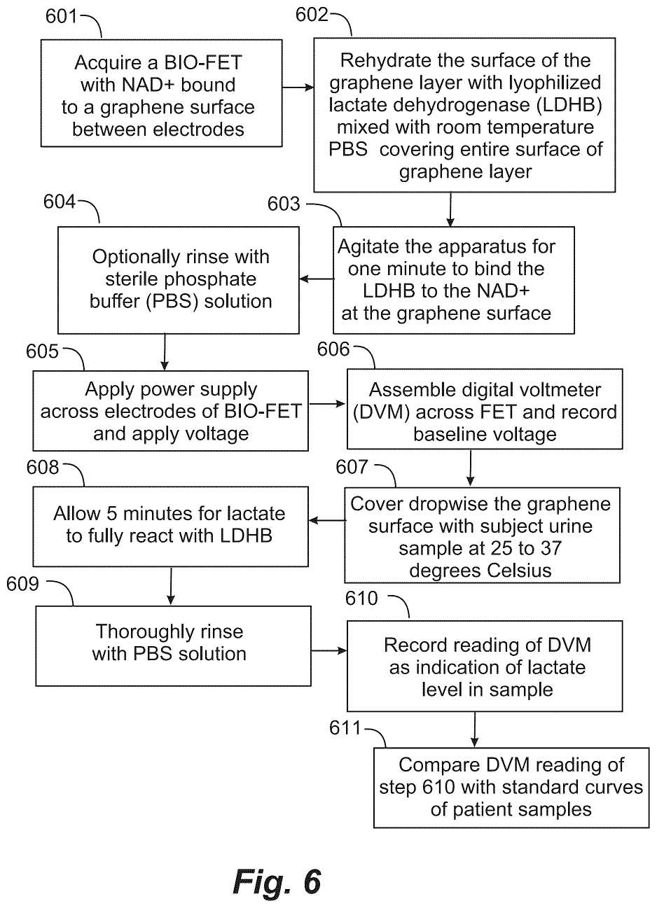

FIG. 6 is a flow diagram of a process for using a prepared BIO-FET to measure lactate in a subject sample. At step 601 a BIO-FET prepared with NAD+ bound to a graphene surface between electrodes is selected. At step 602 the graphene surface is rehydrated with lyophilized lactate dehydrogenase (LDHB) mixed with room temperature PBS. As step 603 the apparatus is agitated for one to three minutes to bind the LDHB to the NAD+. At step 604 the apparatus is optionally rinsed with sterile PBS solution. At step 605 a power supply is connected across the terminals of the BIO-FET and voltage is applied. At step 606 a digital voltmeter (DVM) is connected from the positive terminal of the BIO-FET to the metal bottom layer and the voltage measured is recorded as a baseline voltage. At step 607 the graphene layer is covered dropwise with a subject urine sample at a temperature from 25 to 37 degrees Celsius. At step 608 five minutes is timed for lactate in the subject sample to fully react with LDHB. At step 609 a thorough rinse with PBS solution is accomplished to remove potential impurities from sample that could negatively affect the sensitivity of the device before recording the DVM reading. At step 610 the reading of the DVM is recorded as an indication of lactate concentration in the urine sample. And at step 611 the DVM reading of step 609 is compared with standard curves of patient samples.

In one embodiment a mobile application may be provided for a user to execute on a smartphone, connecting the user's smartphone with a website hosted by a provider of the BIO-FET where coded instructions may be executed to provide interactive interfaces enabling a user to review meanings for the level of lactate determined in a subject urine sample.

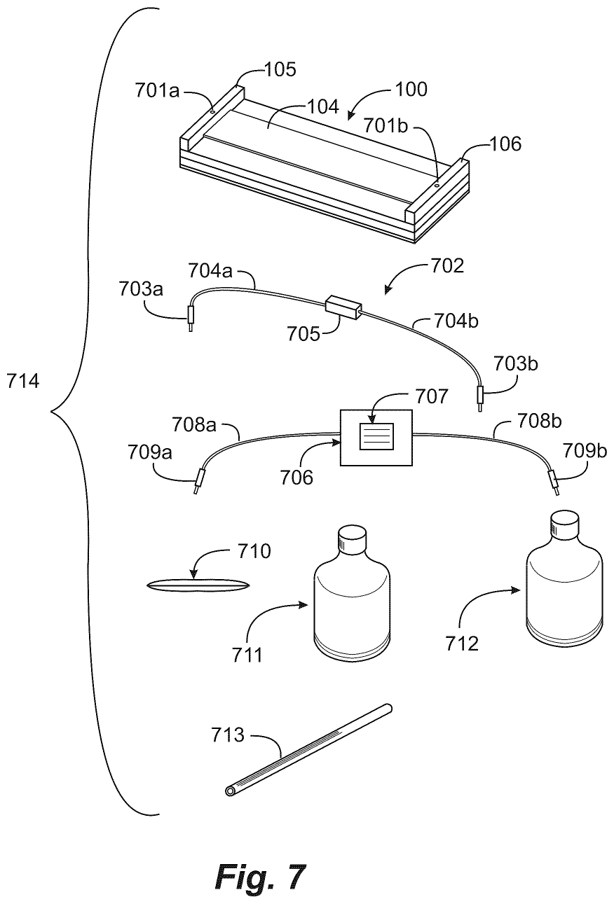

In one embodiment of the present invention a test kit is provided enabling a user to conduct a home urine analysis for lactate concentration. FIG. 7 is an illustration of such a test kit 714 in an embodiment of the invention. Included in kit 714 is a BIO-FET 100 as described in enabling detail above, with the surface of the graphene layer bound with a redox mediator as also described above, ready for use. In this example electrode 105 of the BIO-FET has an opening 701a and electrode 106 has an opening 701b, the openings adapted to accept as a snug fit a pin of a power supply. More than one opening may be included on an electrode to accommodate a pin of a cable of a digital voltmeter.

A power supply 702 is included in the kit with a battery-operated power unit 705 connected by conductor cables 704a and 704b to pins 703a and 703b respectively, the pins adapted to plug into holes 701a and 701b in the electrodes of the BIO-FET. Also included in kit 712 is a digital voltmeter (DVM) 706 adapted especially for the kit, having circuitry and a display 707 with cables 708a and 708b with contact interfaces that may be applied to the BIO-FET across electrode 105 and the metal bottom layer. In one embodiment the contact interfaces may be pins as for the power supply. In other embodiments different contact interfaces may be employed.

A package 710 of LDHB powder is provided, which may be a tear-open packet, or may be provided in a container of a different sort. A small bottle 711 of sterile PBS solution in dionized (DI) water is provided for mixing with the LDHB powder for use in rehydrating the surface of the graphene layer prior to use, as well as a small bottle 712 of sterile PBS solution also for use in rinse procedures in using the kit. Also included is a plastic or glass pipette 713 for use in applying the LDBH solution to the graphene, dropwise, and also for applying a subject urine sample in a test procedure. The pipette may be marked at a position to indicate a specific volume of solution. The pipette may be rinsed between uses with PBS solution.

Kit 712 contents may be packaged in a variety of ways, and the packaging is not described in detail here.

A DIY user having a test kit 712 may essentially follow the flow chart of FIG. 6 to perform a urine test. Instructions may be provided with the test kit and may also be available on a web site accessible by browser, or through a mobile application as described above.

The skilled person will understand that the examples and embodiments described herein are entirely exemplary and do not limit the scope of the invention. The invention is limited only by the claims that follow.

1. A method for preparing a BIO-FET having a graphene layer between a first and a second electrode for a aqueous small molecule assay, comprising:
wetting an exposed surface of the graphene layer with a redox mediator solution; and
allowing time for the redox mediator to bind to the graphene layer.

2. The method of claim 1 wherein the redox mediator is nicotinamide adenine dinucleotide (NAD+).

3. The method of claim 1 further comprising rinsing the BIO-FET with a sterile phosphate buffer solution (PBS), drying the BIO-FET after rinsing, and packaging and storing the dried BIO-FET for later use.

4. A method for conducting a small molecule assay, comprising:
selecting a BIO-FET having a redox mediator bound to an exposed surface of a graphene layer from a stored quantity;
applying an enzyme with sterile phosphate buffer solution (PBS) to the redox mediator-bound graphene layer;
agitating the BIO-FET for a time period, allowing the enzyme to fully bind at the graphene surface;
applying a DC voltage across the first and second electrodes;
applying a digital voltmeter between the first electrode and a metal bottom layer of the BIO-FET;
covering the graphene layer with a solution of a subject small molecule;
allowing a time for lactate molecules in the subject solution to fully react with the enzyme; and
recording a reading of the DVM as an indication of concentration of the small molecule in the subject solution.

5. The method of claim 4 wherein the redox mediator is nicotinamide adenine dinucleotide (NAD+), the solution of a subject small molecule is a subject urine sample, and the enzyme is an isoform of lactate dehydrogenase.

6. The method of claim 4 further comprising, after the agitation step and before the step applying the DC voltage across the electrodes, rinsing the BIO-FET with sterile PBS solution.

7. The method of claim 5 further comprising, after the step selecting a BIO-FET having nicotinamide adenine dinucleotide (NAD+) and before applying a lyophilized lactate dehydrogenase (LDHB) solution, recording a DVM reading as a baseline reading.

8. The method of claim 5 wherein the isoform of lactate dehydrogenase is LDHB.

9. The method of claim 4 further comprising:
executing a mobile application on a cellular telephone;
connecting to a website through the mobile application, the website providing an interactive interface on a display of the cellular telephone enabling entry of the DVM reading; and
entering the DVM reading in the interactive interface.

10. The method of claim 9 wherein, in response to the entry of the DVM reading, information is displayed regarding small molecule concentration in the subject sample, and optional meanings of the concentration.

11. The method of claim 4 further comprising comparing the DVM reading with one or more standard curves.

12. The method of claim 11 further comprising determining by the comparing whether or not that the subject has lactate acidosis, and should be further evaluated.

13. An apparatus for conducting a small molecule assay, comprising:
a field-effect transistor (FET) having a first and a second electrode and a metal bottom layer;
a layer of graphene deposited on an upper surface of the FET between and in contact with the first and the second electrodes;
a redox mediator bound to sites on the graphene layer;
an enzyme bound to the redox mediator on the graphene layer;
a DC power supply adapted to connect across the first and the second electrodes; and
a digital voltmeter adapted to connect across the first electrode and the metal bottom layer of the BIO-FET;
wherein a sample solution containing an unknow concentration of the small molecule applied over the graphene surface causes molecules in the sample to interact with the enzyme, causing a DC voltage to appear across the first electrode and the metal bottom layer, measurable by the DVM as an indication of concentration of the small molecule in the sample solution.

14. The apparatus of claim 13 wherein the redox mediator is nicotinamide adenine dinucleotide (NAD+).

15. The apparatus of claim 14 wherein the enzyme is an isoform of lactate dehydrogenase.

16. The apparatus of claim 10 wherein the DC power supply is battery powered and comprises first and second cables with contact mechanisms adapted to mate with contact mechanisms on each of the first and second electrodes.

17. The apparatus of claim 12 wherein the contact mechanisms on the cables are cylindrical pins and the contact mechanisms on the first and second electrodes are bores with a snug fit to the pins.

18. The apparatus of claim 12 wherein the DVM comprises a first cable with a first contact mechanism adapted to mate with a second contact mechanism at the first electrode, and a second cable with a third contact mechanism adapted to mate with a fourth contact mechanism at the metal bottom layer.

19. The apparatus of claim 14 wherein the contact mechanisms on the cables are cylindrical pins and the contact mechanisms on the first electrode and the metal bottom layer are bores with a snug fit to the pins.

20. A kit for determining concentration of small molecules in a subject sample, comprising:
a biological field-effect transistor (BIO-FET) having first and second electrodes, a metal bottom layer, a layer of graphene deposited on an upper surface of the FET between and in contact with the first and the second electrodes and a redox mediator on the graphene layer;
a battery enabled DC power supply adapted to produce a DC voltage, the DC power supply having a first conductor ending in a first attachment interface, and a second conductor ending in a second attachment interface;
a digital voltmeter having a display, a first cable with a first contact mechanism adapted to mate with a second contact mechanism at the first electrode, and a second cable with a third contact mechanism adapted to mate with a fourth contact mechanism at the metal bottom layer;
a packet of dry enzyme powder;
a capped container of phosphate buffer solution (PBS) mixed with DI water;
a capped container of sterile phosphate buffer solution (PBS); and
a plastic or glass pipette.

21. The kit of claim 20 wherein the redox mediator is nicotinamide adenine dinucleotide (NAD+) and the enzyme is an isoform of lactate dehydrogenase.

22. The kit of claim 16 wherein the pipette is marked at a position indicating a specific volume.

23. The kit of claim 16 further comprising an instruction booklet having instructions for use of the kit in administering a test.
